(19) United States
(12) Patent Application Publication
(10) Pub. No.: US 2010/0087739 A1
(43) Pub. Date: Apr. 8, 2010

(54) APPARATUS FOR OPTICAL BODY ANALYSIS

(75) Inventors: Gerhardus Wilhelmus Lucassen, Eindhoven (NL); Wouter Harry Jacinth Rensen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/442,603

(22) PCT Filed: Sep. 25, 2007
(86) PCT No.: PCT/IB07/53886

§ 371 Date: Mar. 24, 2009

(30) Foreign Application Priority Data
Sep. 26, 2006 (EP) 06300982.3

(51) Int. Cl. A61B 6/00; G01N 21/47; G01J 4/00

(52) U.S. Cl. 600/476; 356/446; 356/364

(57) ABSTRACT

An apparatus for optical body analysis is built with an illumination and detection head, and an optical coupler. The illumination and detection head comprises a light source for illuminating a body portion to analyze through the optical coupler and a detector for receiving light diffusely reflected by the body portion. The optical coupler is mechanically decoupled from the illumination and detection head and is adapted to be in contact with an outer surface of the body portion while the contact between the optical coupler and the body portion minimally affects physical properties of the body portion. The apparatus may further comprise a position unit adapted to adjust the position of the illumination and detection head relative to the optical coupler so that the detector receives through the optical coupler light generated by the light source and diffusely reflected by the body portion.

### FIELD OF THE INVENTION

The invention relates to the field of medical apparatus and more specifically to an apparatus for the optical analysis of body portions such as the lower skin layers. The invention is particularly relevant to the field of non-invasive optical skin analysis for the detection of skin layers properties and components.

### BACKGROUND OF THE INVENTION

Optical measurement of the skin of a patient is often used to detect biological parameters. For instance, Near Infra-Red (NIR) spectroscopy is a non-invasive method for determining glucose concentration in tissue and blood. NIR spectroscopy is often used in diabetes glucose control. It operates as follows: first, a light source illuminates a body portion with NIR light via an optical probe head in contact with skin and light reflected by the body fluids and body tissue is then detected in the probe head.

Patent application JP06-052444 discloses, for instance, a device used to observe an enlarging surface of an examined body by suppressing surface reflected light.

The problem with this type of measure where the probe head is in contact with the skin is that the pressure from the probe head on the skin changes the scattering of light in the skin cellular epidermal, fibrous dermal layer and adipose tissue layer. A drawback of prior art systems is thus that the weight of the probe head affects the physical conditions of the underneath skin layers thereby altering the properties measurements and components concentrations in a non-reliable way.

Therefore, it would be desirable to devise an analysis apparatus that minimally affects the sampling conditions so that, at least, sampling conditions could remain the same for reproducible skin spectral measurements.

### SUMMARY OF THE INVENTION

An object of the invention is to devise an apparatus that minimally alters analysis conditions thereby permitting to achieve reproducible conditions of measurement. To address the above concerns, the invention more precisely relates to an apparatus for optical body analysis comprising first an illumination and detection head, and an optical coupler. The illumination and detection head comprises a light source for illuminating, through the optical coupler, a body portion to analyze and a detector for receiving light diffusely reflected by the body portion. The optical coupler is mechanically decoupled from the illumination and detection head and is adapted to be in contact with an outer surface of the body portion.

The apparatus maintains sampling conditions the same by keeping the probe contact pressure to a minimum. In other words, the contact between the optical coupler and the body portion minimally affects physical properties of the body portion. Indeed, contrary to prior art systems, only the optical coupler is placed on the body portion, e.g. skin surface, and there is little mechanical strength between the optical coupler and the illumination and detection head when such mechanical strength would constrain the head onto the optical coupler. Thus, the apparatus of the invention minimizes the perturbation caused by the overall analysis device to the physical environment of the body portion of interest. In the event the physical conditions are slightly modified due to the weight of the optical coupler or the pressure contact between the optical coupler and the outer layer of the body portion, a physical equilibrium may be quickly established in the lower skin layers and analytes concentrations and physical properties will only be temporarily affected.

In an examplary embodiment, the apparatus further comprises a position unit adapted to adjust the position of the illumination and detection head relative to the optical coupler so that the detector receives through the optical coupler light generated by the light source and diffusely reflected by the body portion.

In an examplary embodiment, the optical coupler is made out of a light weight material. In a particular embodiment, the illumination and detection head further comprises lens(es) arranged in front of the detector to selectively collect light emerging with respect to an area of interest allowing more accurate measurements or shorter measurement times.

In another embodiment, a lens is placed in front of the light source to project the light to the desired area to measure. The lens allows using an extended light source, such as a bulb, rather than a point source, e.g. as a laser, allowing for a safer and lower cost device.

In another embodiment, the light source and the detector are positioned in order to prevent light reflected directly by the optical coupler to enter the detector. This way, only light diffusely reflected by the sample enters the detector. As the light that has been directly reflected off the optical coupler contains no valuable information, this embodiment improves the measurement signal-to-noise ratio. This advantage can also be achieved in another embodiment where each of the light source and the detector comprises a polarizer, the polarization direction of one polarizer being orthogonal to the polarization direction of the other polarizer.

In another embodiment, the optical coupler comprises a chamfer on its edge adapted to prevent direct reflection from the optical coupler to be directed toward the detector.

In a further embodiment, the position unit comprises at least two position sensitive photo detectors adapted to receive light reflected off the chamfer when the illumination and detection head is correctly positioned relative to the optical coupler.

In another examplary embodiment of the invention, the optical coupler is in contact with the body portion through an index matching fluid or gel.

### BRIEF DESCRIPTION OF THE DRAWINGS

These and other aspects of the invention will be apparent from and elucidated with reference to the embodiment described hereafter where:

FIG. 1 is a schematic view of an apparatus according to a first embodiment of the invention;

FIG. 2 is a schematic view of an apparatus according to a second embodiment of the invention;

FIG. 3 is a schematic view of an apparatus according to a third embodiment of the invention;

FIG. 4 is a schematic view of an apparatus according to a fourth embodiment of the invention;

FIG. 5 is a schematic view of an apparatus according to a fifth embodiment of the invention;

FIG. 6 is a schematic view of an optical coupler according to a sixth embodiment of the invention; and

FIG. 7 is a schematic view of an apparatus according to the sixth embodiment of the invention.

In the figures, the same reference number designates a similar or identical object.

### DETAILED DESCRIPTION

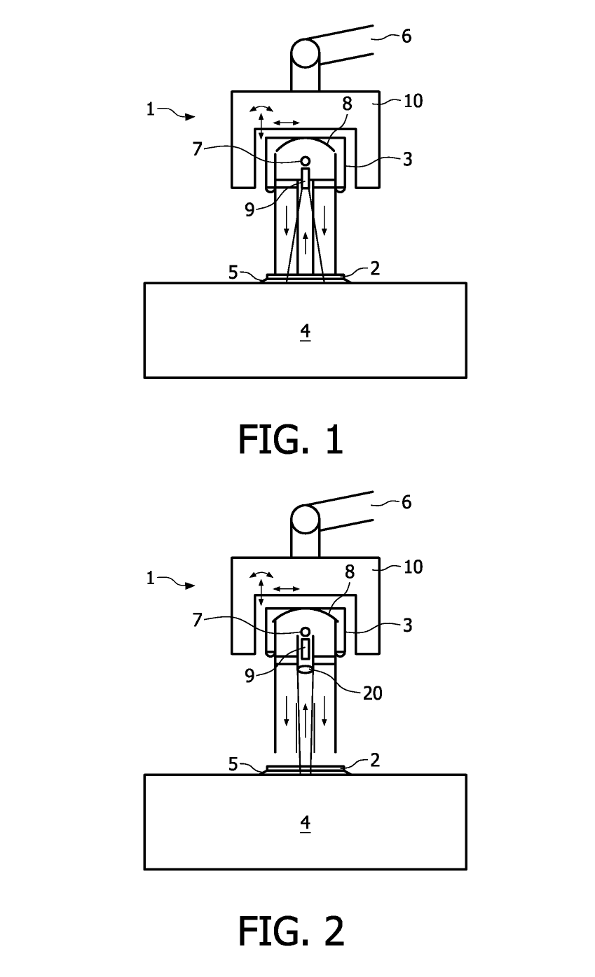

In reference to FIG. 1, an apparatus 1 comprises an optical coupler 2 and an illumination and detection head 3.

The optical coupler 2 is positioned on the outer layer of the body portion 4 to analyze. The outer layer is for example the patient's skin. Optical coupler 2 may be a piece of transparent material with a well defined smooth surface. Optical couplers are typically used to correct for the skin roughness so that the relief of the illuminated surface is known when the skin is illuminated. This makes it easier to predict how much light is reflected and how much light penetrates the skin. Optical coupler 2 can also be either associated with an index matching fluid or gel 5 that is placed between optical coupler 2 and the skin 4 to prevent any air bubbles being trapped at the interface skin-coupler. The index matching fluid 5 minimizes reflection of light passing through optical coupler 2 and the skin 4 or at the interface between the two. The fluid or gel 5 could also be a type of glue that levels out the skin surface 4 while matching the refractive index of the optical coupler 2.

Optical coupler 2 may be made out of a light-weight material so that it applies minimal pressure on the skin surface 4 thereby only minimally affecting the physical condition of the lower skin layers of body portion 4. As previously explained, components concentrations and physical properties like scattering, will thus not be modified at all or only very slightly, yet leading to reliable measurements.

The illumination and detection head 3 is mounted on a support 10. Said support 10 is movable via a position unit 6 which may allow a 6-axis (3 translations, 3 rotations) movement of the head 3. In this embodiment, the illumination portion and the detection portion of head 3 are interdependent, however one could devise an embodiment where the two portions move independently. Also, support 6 may allow less translation and rotational movement, i.e. support 6 could be a 5 or less-axis.

The illumination and detection head 3 comprises a light source 7, such as a bulb or a laser, combined with a reflector 8 to illuminate the skin area. It further comprises a detector 9 such as an optical fiber or a CCD matrix.

The position unit 10 may adjust the position of the illumination and detection head 3 relative to the optical coupler 2 so that the detector 9 receives at least a part of the light generated by the light source 7 after it has been diffusely reflected by the skin area through the optical coupler 2.

FIG. 2 shows another embodiment where a lens 20 is added in front of the detector 9 to more effectively collect light from the location of interest on the sample skin 4. The use of lens 20 permits to better define the surface area from where it is desired to collect diffusely reflected light. Indeed lenses in general more effectively capture light incoming any surface area. The volume that is probed depends on the position of the illumination and detection head 3. In addition, the intensity of the detected diffusely reflect light signal depends on the amount of molecules present in the sampling volume. The knowledge of the sampled volume thus helps in determining the concentration of the molecule of interest. An advantage of this embodiment is that the use of lens 20 may help in the reconstruction of probed sample volume which, as just explained, is a basic parameter for quantitative analysis. A further advantage is that, with the use of the lens 20, a greater fraction of light emerging from the area of interest is captured and launched into the detector 9. As a result, the collected signal level is higher, allowing more accurate measurements, or shorter measurement times.

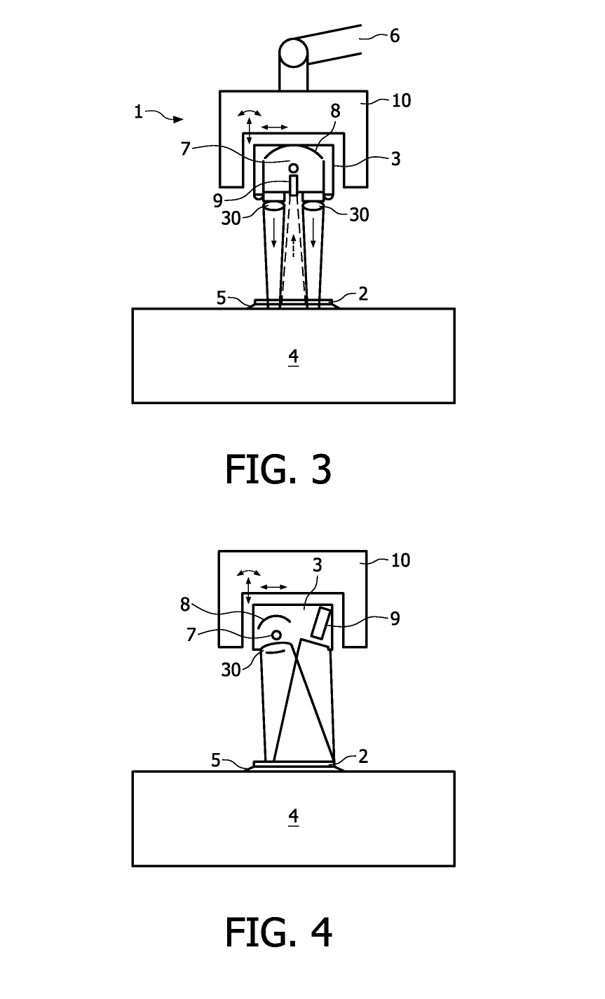

In another embodiment, illustrated in FIG. 3, another lens 30 is included in the head 3 to direct the light from the light source 7 onto a desired area of the sample skin surface 4. A “donut shaped” lens is advantageously used to project light in a shape of a ring on the sample surface 4. It is well known in optical analysis that ring shaped illumination offers a good compromise between light intensity applied to the skin and the illuminated surface leading to optimal results.

The advantage of the lens 30 is that a point source, such as a laser is not necessary therefore allowing for a safer and lower cost device.

In another examplary embodiment, both lenses 20, 30 are integrated as one physical element.

In another embodiment, illustrated in FIG. 4, the light source 7 and the detector 9 are positioned in such a way that light directly reflected off the optical coupler 2 cannot enter the detector 9. For instance, the lens 20 used for illumination and the reflector 8 create a parallel light beam which is incident on the surface of the optical coupler 2 at an angle that does not match the angle of detection of detector 9. This way, only light that has been diffusely reflected by the sample can enter the detector 9. This is an advantage as light directly reflected off the optical coupler 2 does not contain useful information about the skin area 4 and leads to an increased background signal if detected.

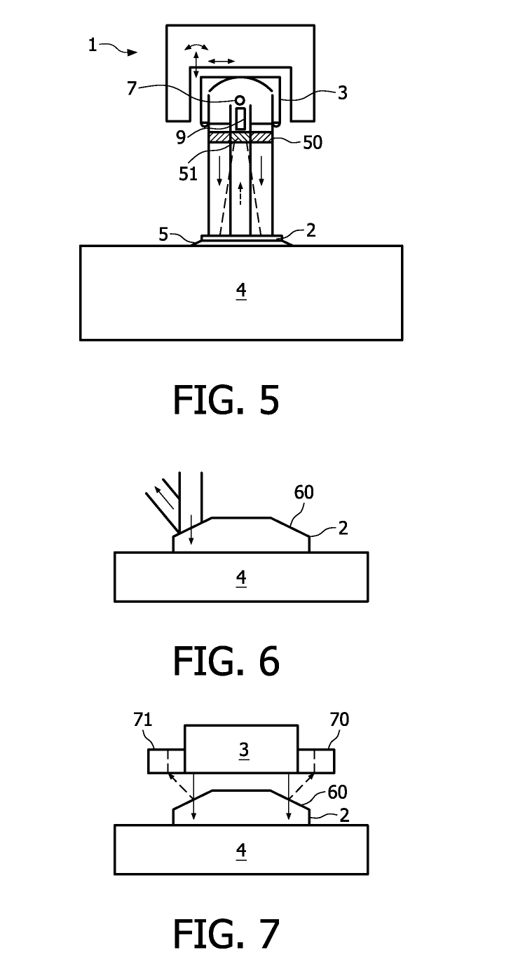

In another embodiment illustrated in FIG. 5, a polarizer 51 is positioned in front of the detector entrance 9 and another polarizer 50 with a polarization direction orthogonal to the first polarizer 51 is positioned in front of the light source 7. This embodiment prevents direct reflection off the optical coupler 2 from entering the detector 9 as direct reflected light has a polarization that is blocked by the polarizer 51. Light that has been diffusely reflected in the skin 4 is depolarized and can partially pass the polarizer 51 in front of the detector 9.

In another embodiment illustrated in FIG. 6, the shape of the optical coupler 2 is adapted to prevent unwanted reflected light from the optical coupler 2 to enter the detector 9. This may be achieved by creating a chamfer 60 on the edge of the upper surface of optical coupler 2.

In an examplary embodiment, illustrated in FIG. 7, the light directly reflected away from the chamfer 60 of the optical coupler 2 may be used to determine the distance between the illumination and detection head 3 and the optical coupler 2. A position sensitive photo detector 70, 71 is added to the head 3, and detector 70, 71 detects the light reflected off the chamfer 60. Once the light reflected off the chamber 60 has been detected, the relative positioning. i.e. vertical distance and horizontal position, of the detection head 30 and the optical coupler 2 may be known. A second set of photo detectors, not shown here, could measure the tilt of head 3 in the direction of the line between the set of detectors 70, 71. A third set of position sensitive photo detectors, also not shown on FIG. 7, may be placed outside the line between the set of detectors 70, 71 to detect tilt in a direction orthogonal to the first tilt direction.

While the invention has been illustrated and described in details in the drawings and foregoing description, such illustration and description are to be considered illustrative or examplary and not restrictive; the invention is not limited to the disclosed embodiment.

For instance, the position unit 6 may provide visual aids to the operator to help him adjust the position of the head 3 or control a motorized support 10 to automatically adjust the position of the head 3.

Other variations to the disclosed embodiments can be understood and effected by those skilled on the art in practicing the claimed invention, from a study of the drawings, the disclosure and the appended claims. In the claims, the word “comprising” does not exclude other elements and the indefinite article “a” or “an” does not exclude a plurality.

1. Apparatus (1) for optical body analysis comprising:
an illumination and detection head (3), comprising:
a light source (7) for illuminating a body portion (4) to analyze through an optical coupler (2), and
a detector (9) for receiving light diffusely reflected by the body portion (4) through the optical coupler (2),
the optical coupler (2) mechanically decoupled from the illumination and detection head (3) and being adapted to be in contact with an outer surface of the body portion (4)

2. Apparatus according to claim 1, further comprising a position unit (6) adapted to adjust the position of the illumination and detection head (3) relative to the optical coupler (2) so that the detector receives through the optical coupler light generated by the light source (7) and diffusely reflected by the body portion (4).

3. Apparatus according to claim 1, wherein the optical coupler (2) is a light-weight coupler that applies minimal pressure onto the body portion (4) when positioned onto the body portion (4).

4. Apparatus according to claim 1, wherein the optical coupler (2) is in contact with the body portion (4) through an index matching fluid or gel (5).

5. Apparatus according to claim 1, wherein the detector (9) comprises at least an optical fiber and a lens in front of said optical fibre.

6. Apparatus according to claim 1, wherein the light source (7) comprises a lens (30) adapted to focus the light generated by the light source on a determined lower skin area of the body portion.

7. Apparatus according to claim 1, wherein the detector (9) comprises a lens (20) adapted to focus the diffusely reflected light from a determined lower skin layer of the body portion (4) onto the detector (9).

8. Apparatus according to claim 6, wherein the light source (7) is positioned around the detector (9) and the lens is donut-shaped.

9. Apparatus according to claim 1, wherein the light source (7) and the detector (9) are positioned to prevent light reflected directly by the optical coupler (2) from entering the detector (9).

10. Apparatus according to claim 1, wherein each of the light source (7) and the detector (9) comprises a polarizer (50, 51), the polarization direction of one polarizer being orthogonal to the polarization direction of the other polarizer.

11. Apparatus according to claim 1, wherein the optical coupler (2) comprises a chamfer (60) on a peripheral edge adapted to prevent light transmitted by the light source and directly reflected off the optical coupler (2) from being directed toward the detector (9).

12. Apparatus according to claim 9, wherein the illumination and detection head (3) comprises at least two position sensitive photo detectors (70, 71) adapted to receive light transmitted by the light source (7) and directly reflected off the chamfer (60).

13. Method to optically analyze a body portion comprising the step of:
positioning an illumination and detection head (3), mechanically decoupled from an optical coupler (2) placed onto an outer layer of the body portion (4), relative to the optical coupler (2), said illumination and detection head (3) comprising at least a light source (7) for illuminating the body portion (4) through the optical coupler (2) and a detector (9), the positioning allowing the detector (9) to receive at least a part of light generated by the light source (7) after it has been diffusely reflected by the body portion (4).
